(19) United States
(12) Patent Application Publication
(10) Pub. No.: US 2026/0276524 A1
(43) Pub. Date: Sep. 17, 2026

(54) PARTICLE CHARACTERIZATION SYSTEM AND METHOD

(71) Applicant: X Development LLC, Mountain View, CA (US)

(72) Inventors: Antonio Raymond Papania-Davis, Oakland, CA (US); Weishi Yan, Oakland, CA (US)

(21) Appl. No.: 19/667,259

(22) Filed: May 4, 2026

Related U.S. Application Data
(63) Continuation of application No. PCT/US2024/050771, filed on Oct. 10, 2024.
(60) Provisional application No. 63/595,657, filed on Nov. 2, 2023.

(51) Int. Cl. G01N 15/1434; G01N 15/14; G01N 33/38

(52) U.S. Cl. CPC G01N 15/1434; G01N 33/383; G01N 2015/1497

(57) ABSTRACT

Methods, systems, and apparatus, including computer programs encoded on computer storage media, for characterizing a particulate ingredient of a mixture including a hopper configured to dispense particles along an axis and in freefall through an imaging region, an illumination sub-system including at least one light source arranged at a first location with respect to the axis and configured to illuminate the imaging region, an image capture sub-system including at least one image capture device including a telecentric lens and arranged at a second location with respect to the axis and configured to align a focal plane of the at least one image capture device with the axis within the imaging region, such that when a particle freefalls through the imaging region, the particle is illuminated by the illumination sub-system and the image capture sub-system captures images of at least three silhouettes of the particle within the imaging region.

### CROSS-REFERENCE TO RELATED APPLICATIONS

This application is a continuation of PCT Application No. PCT/US2024/050771, filed on Oct. 10, 2024, which claims the benefit of priority to U.S. Provisional Application No. 63/595,657, filed on Nov. 2, 2023, the disclosure of these applications are hereby incorporated by reference in their entirety.

### BACKGROUND

Concrete is the second most consumed substance (by mass) on our planet and is responsible for 7-8% of global CO2 emissions. Concrete's material properties are inconsistent due to the large variation in ingredient material (e.g., aggregates) and processing. This material inconsistency requires large safety margins for a given performance level and results in material overuse. Advances in concrete preparation that can optimize the use of locally available materials to maximize concrete performance while minimizing cost with both traditional and non-traditional concrete ingredients are desirable.

### SUMMARY

This specification describes technologies for characterization of particles using optical computational tomography. These technologies generally involve capturing multiple images of a particle at different orientations and using the captured images to rebuild a surface mesh and calculate properties of the particle including, for example, surface area, shape, and volume. Particles can be characterized by a particle analyzer, e.g., in real-time before and/or during a batching process. For example, ingredients, such as powder aggregates, can be monitored by a particle analyzer as they are dispensed from a hopper in a production environment. The particle analyzer employs a multi-frame, short exposure capture of a particle in a stream of falling particles and use computer vision to look at the multiple frames. Computer vision techniques can be used to analyze captured views, e.g., silhouettes, of the particle, define particle boundaries, and characterize particle size, particle size distribution, particle shape, and/or particle surface area. The characterized particles can be associated with a sieve size, where a group of particles can be described by weight percent retained by one or more sieves. Rheometric properties of the mixtures including the particles can be estimated based on the measured characteristics of the ingredient.

In general, one innovative aspect of the subject matter described in this specification can be embodied in methods that include the actions of receiving image data of particles representative of the particulate ingredient of the mixture, where the image data includes sets of frames, and where each set of frames includes silhouettes of one or more particles captured within the set of frames. For each set of frames: identifying, in each frame of the set of frames, a silhouette of at least one particle in the frame, matching respective silhouettes from each frame of the set of frames that represent a same particle appearing in each frame from different perspectives. For the respective silhouettes corresponding to same particle, generating a reconstruction of the particle from the respective silhouettes to obtain a reconstructed particle, and extracting from the reconstruction of the particle, one or more characteristics of the particle. Generating, from the reconstructed particles of the particles, a characteristic profile of the particles, and determining, in response to the characteristic profile, an update to a composition of the mixture.

Other embodiments of this aspect include corresponding computer systems, apparatus, and computer programs recorded on one or more computer storage devices, each configured to perform the actions of the methods.

The subject matter described in this specification can be implemented in particular embodiments so as to realize one or more of the following advantages. The system and methods disclosed herein can be used to characterize a specific surface area per particle in a collection of particles rather than characterizing a specific surface area of the collection of particles, which yields a measurement with reduced variance, e.g., <0.1% variance. A reduced variance can result in an increased confidence of a sieving profile for the collection of particles and a better understanding of the rheological behavior of a composition including the particles, e.g., pack and flow. Reduced variance in measured characteristics can lead to a higher degree of quality control and a narrower six sigma value. Capturing three orthogonal views of a particle can be used to characterize a specific surface area of the particle, which can yield information about the particles, e.g., absorption and/or reaction profiles, flow characteristics, of the particles in a slurry or mixture. By capturing image data of a particle from three (orthogonal) views, the particle can be characterized with a high degree of confidence in the measurement. Understanding rheological properties of mixtures including the characterized particles can be used to predict how the particles will flow and pack in the mixtures. The rheological properties of a resulting mixture including the particles can be better understood such that better predictions can be made about resulting material properties and improved quality control in a production environment, for example, food processing, pharmaceuticals, concrete, or other industries including particles suspends in solution. Understanding the specific surface area can result in better predictions about material properties and improved quality control exiting a production line.

The methods and systems described herein yield improved accuracy in predictions of surface area/volume ratios, in particular, for high-aspect ratio particles which can otherwise be mischaracterized using traditional sieving methods. In some hardware configurations, a single-camera can be used to capture multiple images of a tumbling particle, reducing the hardware cost to implement the characterization system. Additionally, the methods and systems described herein can be used to complement or replace a sieving procedure (set of meshes having different sizes) with a reduced time-requirement computerized procedure, e.g., reduce a time required from about 30 minutes to about 4 minutes or less.

The details of one or more embodiments of the subject matter of this specification are set forth in the accompanying drawings and the description below. Other features, aspects, and advantages of the subject matter will become apparent from the description, the drawings, and the claims.

### BRIEF DESCRIPTION OF THE DRAWINGS

FIG. 1 is a block diagram of an example material preparation system.

FIG. 2 is a block diagram an example particle analyzing system.

FIG. 3 shows a schematic of an example configuration of the particle analyzing system.

FIG. 4 shows a schematic of an example configuration of the particle analyzing system.

FIG. 5 shows a schematic of an example configuration of the particle analyzing system.

FIG. 6 is a flow diagram of an example process of the particle analyzing system.

FIG. 7 is a flow diagram of an example process of the particle analyzing system.

FIG. 8 is a flow diagram of an example process of the particle analyzing system.

FIG. 9 is a schematic diagram of an example computer system.

FIG. 10 shows a schematic of an example particle hopper of a particle analyzing system.

Like reference numbers and designations in the various drawings indicate like elements.

### DETAILED DESCRIPTION

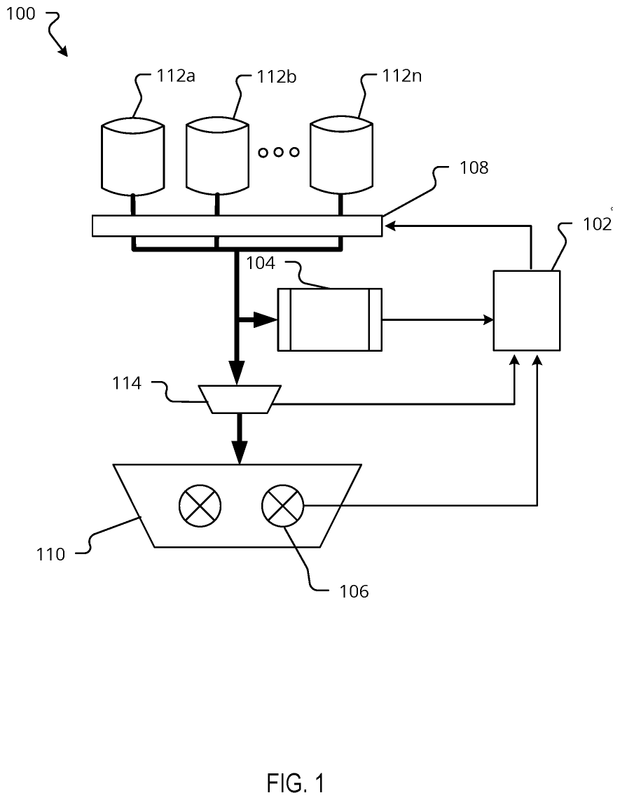

FIG. 1 shows an example material preparation system 100. Material preparation system 100 can be, for example, a concrete preparation system, slurry preparation system, or another material composition preparation system. In operation, material preparation system 100 measures characteristics of one or more of the raw ingredients that are included into the material mixture. System 100 can adaptively adjust proportion(s) of the raw ingredients added into the mixture based in part on measured characteristics to achieve a set of target properties of the mixture and/or final product more accurately. Target properties of the mixture can include, for example, rheological properties (e.g., flow characteristics) of the mixture. Target properties of the final product can include, for example, structural properties, electrical properties, thermal properties, chemical properties, or the like, of a final product.

Material preparation system 100 includes a control system 102. The control system 102 receives input from particle analyzing system 104 and composition mix sensors 106. The control system 102 can control the operations of one or more ingredient metering systems 108 based on analyses of data obtained from one or both of the particle analyzing system 104 and composition mix sensors 106.

Material preparation system 100 includes raw ingredient storage bays or hoppers 112a-112n. The ingredient metering system 108 conveys the raw ingredients from the storage bays 112a-112n to a mixing vessel 110. For example, the ingredient metering system 108 can include a series of conveyors and augers to transfer raw ingredients from the storage bays 112a-112n into the mixing vessel 110.

In some embodiments, at least a portion of the raw ingredients, either individually or as a mixture, are passed through the particle analyzing system 104 prior to delivery to the mixing vessel 110. As depicted in FIG. 1, a sample portion of the raw ingredients are separated into a secondary stream and provided for characterization to the particle analyzing system 104 to perform real-time characterization of the sample portion of the raw ingredients during a mixing/batching process. The sample portion of the raw ingredients can be drawn off without disrupting an active production line and the measured characteristics of the sample portion can be used to provide feedback to the control system 102 for updating a composition of the mixture, as described in further detail below.

In some embodiments, particle analyzing system 104 can receive a sample portion of one or more of the raw ingredients to characterize prior to initiating a mixing/batching process, where the measured characteristics of the sample portion can be provided to the control system 102 and input into a batching recipe for production of the mixture.

In some embodiments, particle analyzing system 104 can be in-line with a primary stream of material within system 100 where particle analyzing system 104 can characterize raw ingredients prior to entry into mixing vessel 110.

In some implementations, the ingredient metering system 108 may include a metering hopper 114 along the primary process stream and prior to the mixing vessel 110. For example, the weight of the ingredient measured by metering hopper 114 can be passed to the control system 100 permitting the control system to monitor the weight of the ingredient being measured in real-time. The control system 102 may then be able to make in-situ adjustments to how much of the ingredient to add to the concrete mixture based on real-time particle analysis of the ingredient from the particle analyzing system 104. In some implementations, material preparation system 100 can be retro-fit to a traditional production environment, e.g., into a traditional ready-mix concrete plant. For example, adding the material preparation system 100 to a ready-mix plant may allow the ready-mix plant to more precisely tailor concrete mixes for specific applications and job sites.

Particle analyzing system 104 can include various different sensors configured to measure various characteristics of the composition ingredients. For example, the sensors used by the particle analyzing system 104 can include, but are not limited to, optical sensors (e.g., visible light cameras, infra-red cameras, dynamic optical microscopy sensors) and mechanical sensors (e.g., sieves, sedigraphs, impact hammer, electrodynamic vibrator). The measurement data is used by control system 102 to determine characteristics of the ingredients of the mixture. For example, ingredient characteristics can include, but are not limited to, particle size, particle shape, particle volume, specific surface area, and particle sphericity. Additionally, particle analyzing system 104 can include various different sensors configured to measure distributions of characteristics across one or more ingredients included in a composition. For example, particle size distributions, specific surface area distributions, particle volume distributions, etc. The optical sensors can transmit images of the ingredients to control system 102, which (as explained in more detail below) can use image processing algorithms to identify particle shapes and sizes.

Mixture sensors 106 provide rheometric measurements of the mixture to the control system 102. For example, the mixture sensors 106 can measure various attributes of the mixture that can be used to estimate or compute rheological properties of the mixture in real-time. Mixture sensors 106 can include, but are not limited to, viscosity sensors, rheometers, temperature sensors, moisture sensors, ultrasonic sensors (e.g., ultrasonic pulse velocity sensors), electrical property sensors (e.g., electrodes, electrical resistance probes), electromagnetic sensors (e.g., short-pulse radar), or other sensors (e.g., geophone, accelerometer). Mixture sensors 106 can include, but are not limited to, hydrophobicity, moisture content, XRD spectra, XRF spectra, static yield stress, acoustic impedance, p-wave speed, dynamic yield stress, static modulus of elasticity, Young's modulus, bulk modulus, shear modulus, dynamic modulus of elasticity (DME), Poisson's ratio, density, resonance frequency, nuclear magnetic resonance (NMR), dielectric constant, electric resistivity, polarization potential, and capacitance. As described in more detail below, control system 102 can use measured characteristics for the raw ingredients and mixture to predict properties of a final product or mixture, and, in response, update a batching recipe to obtain target material properties.

In some implementations, particle analyzing system 104 is configured to perform optical computational tomography of a stream of particles. Particle analyzing system 104 can be configured to capture images of a stream of particles, where the images capture one or more perspectives of a particle within the stream of particles. Control system 102 can receive the captured images and extract, from the images, particle characteristics of the particle in the stream of particles, e.g., specific surface area, particle volume, particle shape, etc., which can be used to predict behavior (e.g., flow characteristics, packing density, etc.) of a mixture including the particles of the stream of particles.

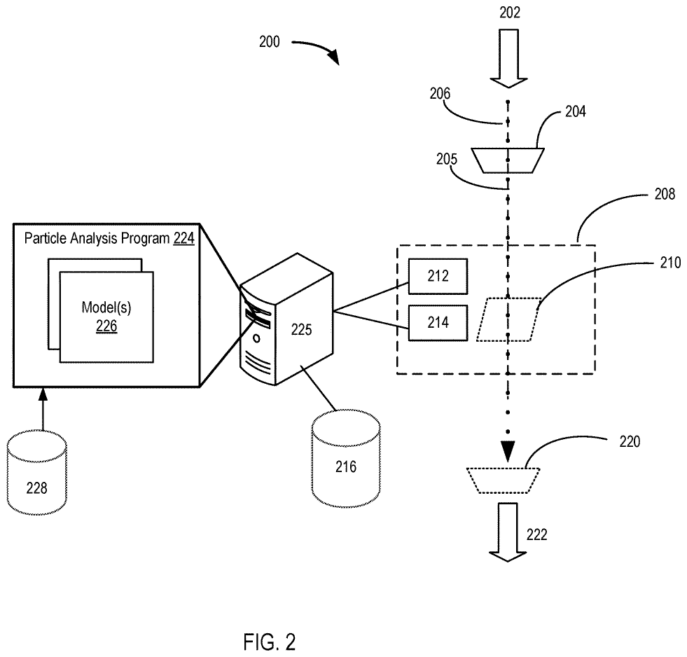

FIG. 2 depicts an example particle analyzing system 200. As described above, particle analyzing system 200 can be configured to capture real-time data as a sub-system of a material preparation system, e.g., material preparation system 100. A portion of the raw ingredients can be diverted into a secondary stream and provided to the particle analyzing system 200. In some implementations, particle analyzing system 200 can be a sub-system along a primary stream of ingredients in the material preparation system.

In any case, particle analyzing system 200 is configured to receive 202 a volume of raw ingredient, e.g., particles, into a hopper 204. Hopper 204 is configured to generate a freefall stream of particles 206 through an imaging region 208. In some implementations, Hopper 204 can include a slot to generate a controlled flow of particles exiting the hopper 204 and into the imaging region 208.

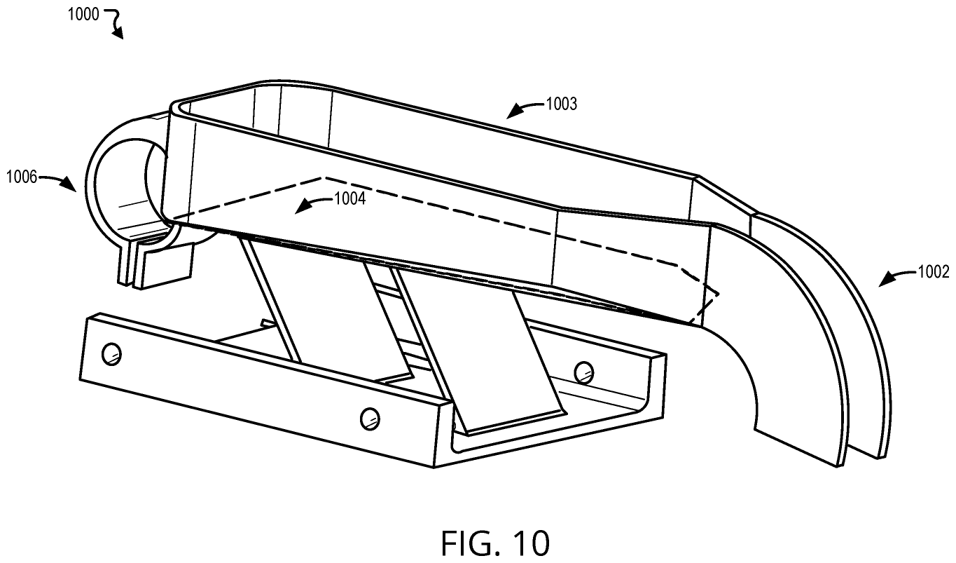

In some implementations, for example, as depicted in FIG. 10, a hopper 1000 includes a slot 1002 to generate a controlled flow of particles exiting the hopper 1000. Additionally, hopper 1000 includes a reservoir 1003 optionally including slanted plane 1004 to retain particles in the reservoir and direct a flow of particles along the plane 1004 into the slot 1002. The hopper 1000 can include an agitator component 1006 which can be coupled to an agitator, e.g., a motor, ultrasonic source, etc., which can be configured to induce vibratory or oscillatory motion in the slanted plane 1004 and cause particles on the slanted plane 1004 to flow towards the slot 1002. In some implementations, hopper 1000 can be coupled to a secondary particle reservoir (not shown) that is configured to retain particles and dispense the particles into the hopper 1000 in a controlled manner.

In some implementations, the freefall stream of particles 206 is a single-width particle stream along an axis 205. In other words, the freefall stream of particles forms a substantially one-dimensional sheet intersecting a plane 210 in the imagining region 208. For example, a single particle crosses plane 210 that is oriented perpendicular to the axis 205 of particle stream 206 in the imaging region 208 at a time T during a dispense of the freefall stream of particles 206. As depicted in FIG. 2, stream of particles 206 includes a freefall portion through at least the imaging region 208, where freefall is an unguided dispense of the particles, e.g., a waterfall or cascade of particles unsupported by a physical guide or rail.

In some implementations, particle stream 206 can be collected by a collection hopper 220. Particles collected in collection hopper can be reintegrated into the primary process 222 of the material preparation system 100. In some instances, particles collected in the collection hopper can be further analyzed, e.g., through a sieving process or another characterization process.

Particle analyzing system 200 includes an image capture sub-system 212 and an illumination sub-system 214. Illumination sub-system 214 is configured to illuminate (e.g., backlight) particles of the particle stream 206 within imaging region. Image capture sub-system 212 is configured to capture image data 216 of particles of the particle stream 206 within imaging region 208. A focal plane of one or more image capture devices (e.g., cameras) of the image capture sub-system 212 can be aligned with the flow of the particle stream through imaging region 208.

Illumination sub-system 214 includes one or more light sources, e.g., light emitting diodes (LEDs), bright light sources, diffuse light sources, or the like. The light sources of the illumination sub-system 214 are arranged with respect to the particle stream 206 to provide a back-lit (e.g., high contrast) condition to the particles being imaged by the image capture sub-system 212. At times, the light sources can emit different wavelength spectra (e.g., different colors of light). In some implementations, the illumination sub-system 214 can include filters. For example, a color filter or polarizing filter, arranged with respect to each light source. The emitted light signal from each filtered light source can be distinct from each other filtered light source. For example, each filtered light source can have a distinct wavelength spectrum (e.g., red, green, blue). In another example, each filtered light source can have a distinct polarization of light (e.g., different angular orientations).

The image capture sub-system 212 includes one or more cameras. The one or more cameras can include respective telecentric lenses. A telecentric lens can have, for example, a magnification 0.05-0.055× magnification that is constant across the depth of field of the telecentric lens. At times, for example, for a known distance between camera and the flow of particles, (e.g., when a particle flow is along a ramp), the cameras can be implemented in the image capture sub-system without a telecentric lens. In such instances, the image capture sub-system 212 can implement an image segmentation network.

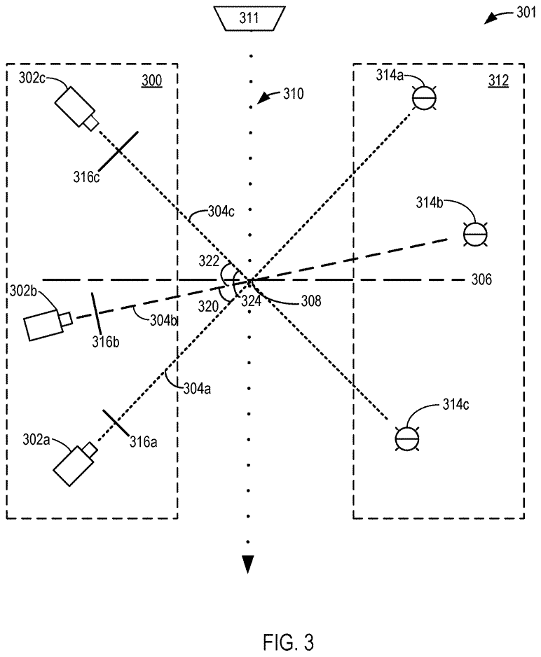

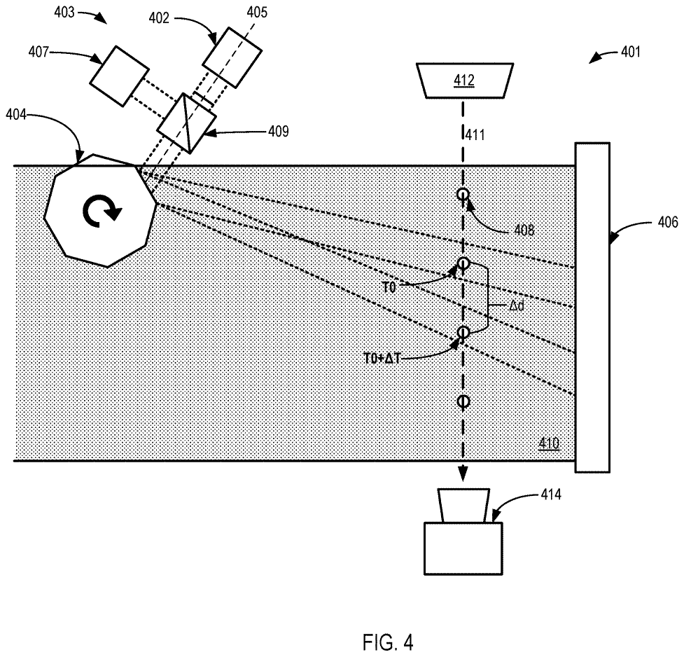

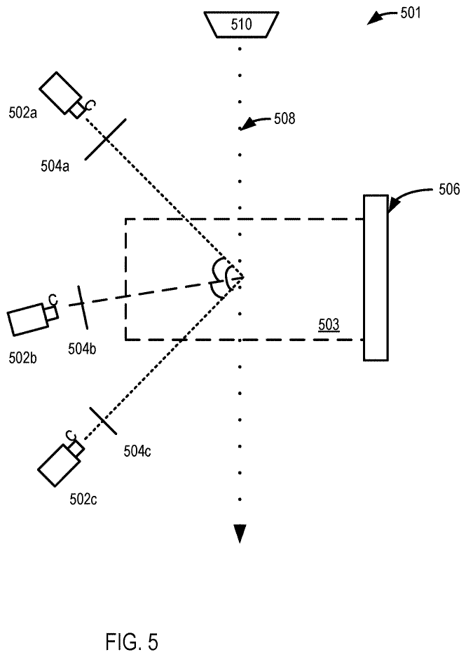

The image capture sub-system 212 can be configured for stationary image capture, e.g., as described with reference to FIG. 3, or can be configured to scanning image capture, e.g., as described with reference to FIGS. 4-5. The image capture sub-system 212 is configured to capture multiple views of each particle of a set of particles of the particle stream. A view of the particle of the particle stream 206 within a captured frame includes a threshold contrast between the particle and the background. For example, the captured frames capture a view, e.g., a silhouette, of the particle within the frame.

The multiple views of each particle of the set of particles can be captured by a single-camera or a multi-camera configuration. In the case of a single-camera configuration, as described in further detail below with reference to FIG. 4, multiple views of the particle are captured as the particle freefalls (e.g., tumbles) through the imaging region and where each view is captured at a different time T. The resulting number of views of the particle captured by the single camera can include up to the number of frames captured by the single camera while the particle is freefalling through the imaging region (e.g., N frames). In a first case of a multi-camera configuration, multiple views of the particle are captured as the particle freefalls through an imaging plane within the imaging region, where each camera of the multi-camera configuration captures a synchronized frame of the particle at the imaging plane, e.g., as described in further detail below with reference to FIG. 3. A resulting number of views of the particle captured by the multiple cameras can include up to the number of cameras in the configuration, e.g., M views where Mis a number of cameras. In a second case of a multi-camera configuration, multiple views of the particle are captured by each camera of the multi-camera configuration, where the multi-camera configuration captures multiple sets of synchronized frames, e.g., as described in further detail below with reference to FIG. 5. A resulting number of views of the particle captured by the multiple cameras can include up to the number of cameras and the number of frames per camera, e.g., NM views where N is a number of frames per camera and M is a number of cameras.

In some implementations, the one or more cameras of the image capture sub-system 212 are aligned with the flow of the particle stream such that a field of view of each camera is oriented with respect to the axis 205 of the particle stream 206 to minimize a likelihood of one particle being at least partially obscured or occluded behind another particle or where two overlapping particles can appear as a single particle in a captured image. For example, a field of view of each camera of the one or more cameras of the image capture sub-system can be oriented orthogonal to the axis 205 of the particle stream 206. Example configurations of image capture sub-system 212 and illumination sub-system 214 are described in further detail with reference to FIGS. 3-5 below. Operation(s) of image capture sub-system 212 and illumination sub-system 214 can be controlled by a control system, e.g., control system 102, hosted on one or more servers 225.

At times, particle analyzing system 200 can be configured to capture fewer than each particle in particle stream 206 in freefall through the imaging plane 210. Shutter speeds of the cameras of the image capture sub-system 212 can be set to capture a threshold subset of the particles dispensed by hopper 204 while maintaining a threshold clarity and low distortion of the images capturing the subset of particles. For example, cameras can be set to capture image data (e.g., frames) at about 50 milliseconds intervals.

Image data 216, e.g., the captured camera frames by the one or more cameras of the image capture sub-system 214, can include one or more particles of the particle stream within each captured frame. Image data 216 captured by image capture sub-system 212 is provided to a particle analysis program 224. Particle analysis program 224 can be hosted on one or more servers 225, e.g., one or more servers of a control system 102. The particle analysis program 224 can receive the image data 216 as input and provide, as output, one or more characteristics of the particles characterized from the particle stream. The one or more characteristics include, for example, specific surface area, volume, aspect ratio, or the like. The particle analysis program 224 can provide, as output, one or more predictions of material composition properties including the characterized particles of the particle stream. For example, rheological properties including, e.g., flow characteristics, packing density, and the like. The particle analysis program 224 can provide, as output, characterization of the particles of the particle stream in terms of a sieving profile, e.g., predictions of what the sieving profile is for the particles of the particle stream without requiring performing the sieving process.

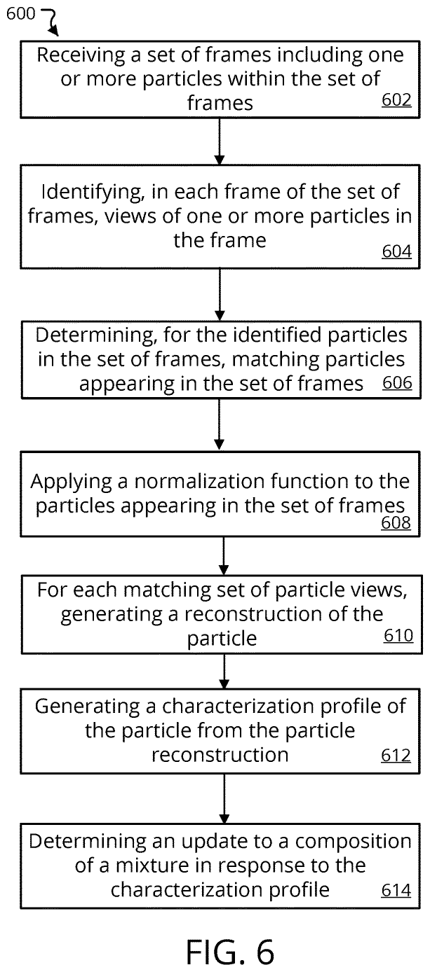

In some implementations, particle analysis program 224 includes one or more models 226, e.g., one or more machine-learning models, which can be implemented by the particle analysis program to generate predictions from the image data 216. The one or more machine-learning models 226 can be trained using training data 228. Further details of the operations of the particle analysis program are described with reference to FIGS. 6-8 below.

The one or more outputs of the particle analysis program 224 of the particle analysis system 200 can be provided as input to the material preparation system 100. The material preparation system 100 can receive the one or more outputs, e.g., characteristics of the particles in the particle stream, and implement one or more adjustments in response. For example, batch recipes, batch ingredient ratios, preparation methods, or the like, can be adjusted in response to the characterization of the particles of the particle stream output by the particle analysis system 200.

### Stationary Image Capture

In some implementations, image capture sub-system 212 includes two or more cameras oriented to capture two or more different views of particles in the freefall particle stream 206. FIG. 3 shows a schematic of an example configuration of the particle analyzing system 301. As depicted in FIG. 3, an image capture sub-system 300 includes three cameras 302a, 302b, and 302c, including telecentric lenses. The telecentric lenses can be, for example, bi-telecentric lenses. Each of cameras 302a-c is oriented orthogonally with respect to each other of the cameras, i.e., such that a respective axis 304a, 304b, and 304c aligned with the cameras 302a, 302b, and 302c, respectively, will form a corner of a cube at the imaging plane 306. Cameras 302a-c are arranged to align a focal plane of each camera with a point 308 on the imaging plane 306 where particle stream 310 from hopper 311 intersects with the imaging plane 306.

Particle analyzing system 301 includes an illumination sub-system 312 including three light sources 314a, 314b, and 314c. Light sources 314a-c can be, for example, light-emitting diodes (LEDs) or another point light source. Light sources 314a-c can emit the same emission spectrum (e.g., white light sources) or light sources 314a-c can have different emission spectra, e.g., red, green, and blue. Each light source 314a-c is arranged along an axis 304a, 304b, 304c with respect to a respective cameras 302a-c.

In some implementations, image capture sub-system 300 includes filters 316a, 316b, 316c arranged with respect to each camera 302a, 302b, 302c of the image capture sub-system 300. For example, light sources 314a-c can each emit (or be filtered to emit) a respective emission spectrum (e.g., red, green, blue) or distinct polarization, and cameras 302a-c can each include a respective filter to select for the respective emission spectrum or distinct polarization of the camera 302a, 302b, 302c. In this way, each camera 302a-c captures only light emitted from a corresponding light source 314a-c aligned along a respective axis 304a, 304b, 304c.

The three cameras 302a-c of image capture sub-system 300 are arranged to capture three orthogonal views of a sample particle of the particle stream 310 as the particle freefalls through imaging plane 306. Cameras 302a and 302b are arranged at an angle 320 with respect to each other. Cameras 302b and 302c are arranged at an angle 322 with respect to each other. Cameras 302a and 302c are arranged at an angle 324 with respect to each other. As depicted in FIG. 3, angles 320, 322, and 324 are each 90 degrees, such that a respective axis 304a-c of each camera intersects the imaging plane 306 at 45 degrees (e.g., 45 degrees with respect to the particle stream 310). As the particle freefalls through imaging plane 306, the particle is illuminated by each of light sources 314a-c such that the respective camera 302a-c captures image data including a shadow of the particle (e.g., a silhouette) from the perspective of the camera 302a-c. Cameras 302a-c can be configured to capture image data with a short exposure time (e.g., 20-30 milliseconds) to reduce blurring of the images and distortion, e.g., due to the movement of the particle stream.

Though depicted in FIG. 3 as orthogonal (90 degree) angles between the cameras 302a-c, angles 320, 322, and 324 between cameras 302a-c can be less than 90 degrees. For example, the values of angles 320, 322, and 324 can range between 80 and 90 degrees. In another example, the values of angles 320, 322, and 324 can range between 82-85 degrees.

In some implementations, a particle analyzing system can include more or fewer cameras than the configuration described with reference to FIG. 3. For example, a particle analyzing system can include two cameras or four cameras. At times, a number of cameras included in a particle analyzing system can depend on a threshold relative accuracy requirement of characteristics of the particles in the particle stream. An accuracy requirement can be, for example, a variance in capturing information of a population of particles. For example, increasing a number of cameras (e.g., 4 or more cameras) may provide an increase in relative accuracy of measurements by the particle analyzing system over a three-camera configuration, whereas a decrease in number of cameras (e.g., 2 or fewer cameras) may provide a decrease in relative accuracy over the three-camera configuration, e.g., a linear relationship between a variance in the measured properties of a population of particles. At times, a number of cameras to include in the particle analyzing system depends on desired characteristics to measure of the population of particles using the system. For example, using the system to measure a gradation (e.g., a weighted histogram) of the population or to generate predictions related to how the particle population will affect material properties of a material composition including the particle population, may benefit from reducing a variance of the measurements such that a value of including additional cameras (e.g., 3, 4, or more cameras) is beneficial.

At times, a number of cameras included in a particle analyzing system can depend on characteristics of the particle stream dispensed by a hopper of the system. For example, in configurations where the particle stream is dispensed as a two-dimensional curtain, a two-camera configuration may be selected for the particle analyzing system.

### Scanning Image Capture Configuration

In some implementations, a camera of particle analyzing system can capture multiple frames of a particle at different locations along an axis defined by the particle freefall that includes different perspectives of the particle. The multiple frames can be, for example, three or more frames, four or more frames, five or more frames. The particle in freefall may additionally include a tumbling, e.g., rotational, motion with respect to an axis of freefall, such that the camera can capture one or more views of the particle as it tumbles through the imaging region of the particle analyzing system. For example, an image capture sub-system can include a scanning system, e.g., a polygon mirror or a galvo mirror scanning system, which can track the particles through an imaging region by adjusting the field of view of the camera using the scanning system.

FIG. 4 shows a schematic of an example configuration of a particle analyzing system 401. Image capture sub-system 403 includes camera 402 having a telecentric lens and is aligned with the rotating faces of a polygon mirror 404 along an axis 405. Optionally, image capture sub-system 403 includes a line scanner 407 and a beam splitter 409 arranged with respect to axis 405 such that the line scanner is configured to capture high speed measurements of the particle 408 in free fall. Data captured by line scanner 407 can be used to increase a temporal precision of the captured image data from camera 402 by correcting motion-based distortion.

Particle analyzing system 401 includes a bright diffuse backlight 406 to provide illumination for the camera 402 of the particle analyzing system. Diffuse backlight 406 has at least a threshold brightness to illuminate a particle stream including particle 408 as the particle free falls through imaging region 410 along axis 411.

Particle analyzing system 401 includes a metered particle dispenser, e.g., hopper 412. Hopper 412 can include a tumbling feature configured to induce a tumbling (e.g., rotational) motion in a particle 408 as the particle is dispensed by the hopper 412 into the imaging region 410. For example, a vibrational and/or oscillatory motion can be applied to the hopper by a motor coupled to the hopper, such that a tumbling motion is induced into the particles retained by the hopper 412. A metered dispense of particles by hopper 412 can be controllable, e.g., by control system 102, to dispense one or more particles at a time through the imaging region and to maintain at least a threshold distance between two particles through the imaging region. For example, control system 102 can be operable to control a timed dispense of particles (e.g., N particles per unit time) by operating a shutter or flap of hopper 412 with a selected interval. Particle collector 414 collects particles of the particle stream and can optionally return the collected particles into a composition mixture process, e.g., a primary stream of system 100.

In some implementations, a control system 102 is operable to perform time-division multiplexing of the collected image data. The control system can synchronize a dispense of a particle 408 from hopper 412 into imaging region 410 with collection of image data by image collection sub-system 403. In other words, a rotational speed of the polygon mirror 404 and frame capture speed of the camera 402 are synchronized and selected to capture a threshold number of frames of a particle 408 within imaging region 410, e.g., at least five frames. For example, a first frame of particle 408 at a first position along axis 411 in imaging region 410 can be captured at T0 and a second frame of particle 408 at a second position along axis 411 is captured at T0+ΔT, where ΔT is about 50 milliseconds and where a distance Δd is a distance along axis 411 traveled by the particle during time ΔT. For particles having horizontal speeds that are negligible (e.g., nearly or approximately zero), and that are within the imaging region 410, vertical speeds of the particles are approximately (e.g., within a threshold range) the same. Using a previous position captured of a given particle in a captured frame and speed of the given particle, a dead reckoning technique can be used to predict and track which images of a particle corresponds to the given particle in subsequent frames.

At times, the speed at which individual particles freefall through imaging region 410 can differ, e.g., based on size, shape, orientation, etc., of the different particles. The variation in speeds can result in distortion of the image data captured of the different particles, e.g., due to the frame capture rate not exactly aligning with position of the particle at different position in freefall through the imaging region 410. Data captured by line scanner of the imaging region 410 through the polygon scanner can be used to compensate for the distortion caused by the variation in freefall speeds in the silhouette images of the particles. For example, a Δt of the line scanner can be less than about ≤10-5 seconds such that a velocity is calculated for each freefalling particle at

$$v = \frac{Δ d}{Δ t}$$

to compensate for the distortion at time t. A calibration process to compensate for distortion can include using a calibration object (e.g., a ball bearing) of known dimensions/weight and scan the calibration object under the telecentric conditions to correct the distortion.

In some implementations, particle analyzing system can collect image data for frequency-division multiplexing using different frequency channels (e.g., color) to distinguish frames of a same particle captured at different positions within the imaging region during freefall of the particle. In such cases, diffuse light source includes different color light sources (e.g., red, green, blue) and image capture sub-system includes corresponding color filters (e.g., filters 316a-c as depicted in FIG. 3). During image capture of the particle, the rotating scanner system captures a different filtered light emission at different locations within the imaging region, by switching a filter along with the rotation of the polygon scanner.

Although described with reference to FIG. 4 with respect to one camera 402, particle analyzing system 401 can include two or more cameras, e.g., 3 cameras, 4 cameras, etc., similarly configured, e.g., as depicted in FIG. 5, where each camera captures a set of frames of the particle as the particle freefalls through the imaging region.

FIG. 5 shows a schematic of an example configuration of a particle analyzing system 501. Each of the image capture sub-systems 502a, 502b, and 502c are similarly configured as image capture sub-system 403 described with reference to FIG. 4, where each image capture sub-system 502a-c is configured to capture multiple frames of a tumbling particle through the imaging region 503. Each of the image capture sub-systems 502a-c includes a camera, scanner system (e.g., a polygon or galvo mirror scanner system), and optionally a beam splitter and line scanner for generate line scan data for the freefalling particles. Each of the image capture sub-systems 502a-c can collect image data over a range of angles based on an angular extent of the scanner system. The cameras of the image capture sub-systems 502a-c are arranged at respective angles from each other camera, as described with reference to FIG. 3. For example, the cameras of the image capture sub-systems 502a-c can be positioned at orthogonal (90 degree) angles between the cameras. In another example, the cameras can be positioned at angles of less than 90 degrees between the cameras, e.g., between 80 and 90 degrees, or between 82-85 degrees.

Image capture sub-systems 502a-c include a respective filter 504a, 504b, and 504c, where each filter 504a-c filters a distinct emission spectrum (e.g., red, green, blue) or a polarization of the light from a diffuse source 506, such that image capture sub-systems 502a-c capture silhouette(s) of the particle in distinct channels. As configured with respect to FIG. 5, each of the image capture sub-systems 502a-c capture multiple frames of a particle in a particle stream 508 dispensed by hopper 510. The multiple frames track the particle as the particle freefalls through the imaging region 503.

In some implementations, hopper 510 is configured to induce a tumbling motion in a particle of the particle stream, such that each camera of the cameras 502a-c captures different perspectives of the particle as the particle freefalls through the imaging region 503. Thus, for a particle of the particle stream that is captured in the image data by the cameras 502a-c, the particle analyzing system generates multiple views of the particle captured from three different camera angles, which can be used to determine characteristics of the particle, e.g., as described in further detail with reference to FIG. 6.

In some implementations, diffuse source 506 includes multiple single color light sources (e.g., single color LEDs) arranged with respect to the diffuse source 506 to illuminate the particle stream 508 dispensed by a hopper 510. Filters 504a-c are color filters, where each filter selects a different color of light emitted by the diffuse source 506.

### Particle Analysis Methods

As described above, a particle analyzing system is used to characterize particles from collected image data. The methods of the particle analyzing system can be performed on a representative sample of raw materials processed in system 100. For example, a continuous monitoring process can include diverting a representative sample of raw material(s) from a primary stream of system 100 to characterize the material(s). In another example, a representative sample can be collected from a batch of raw material(s) to characterize the batch in an intermittent (e.g., once-a day) characterization of raw material(s).

In some configurations, the image capture sub-system of the particle analysis system includes a stationary image capture, where N frames are captured as the particle crosses an imaging plane perpendicular to the freefall axis, where N is the number of cameras in the image capture sub-system, e.g., as described with reference to FIG. 3. In some configurations, the image capture sub-system includes a scanning image capture, where NM frames are captured as the particle freefalls through an imaging region parallel to the freefall axis, where M is the number of cameras and N is the number of consecutive frames captured (e.g., ~50 milliseconds apart) by each camera of the particle as the particle freefalls through the imaging region, e.g., as described with reference in FIGS. 4 and 5. The captured images include N synchronized image sets, each synchronized image set including N frames captured at a synchronized time stamp by M cameras in the system.

In any case, a subset of frames captured by the image captured sub-system include views (e.g., silhouettes) of one or more particles within the frames which can be analyzed and processed to generate predictions of characteristics of the one or more particles. Within the subset of frames, the frames include a number of particles captured from the respective fields of view of the one or more cameras, where a threshold number of particles captured in the respective fields of view of the one or more cameras are the same particles. For example, at least one particle in the subset is common to all the frames in the image subset.

FIG. 6 shows a flow diagram of an example process 600 of the particle analysis system. For convenience, the process 600 will be described as being performed by a system of one or more computers, located in one or more locations, and programmed appropriately in accordance with this specification. For example, a control system 102, appropriately programmed, can perform the process 600. Additionally, for clarity, the following process will be described with respect to an image capture sub-system having a three-camera, stationary configuration (e.g., as depicted in FIG. 3). However, the methods described with reference to FIG. 6 can be applied to other numbers of cameras in a particle analysis system and/or a scanning camera configuration providing at least two views of the particle stream, where at least two views of a particle are captured by the image capture sub-system.

The particle analysis program, e.g., particle analysis program 224, receives a set of frames, e.g., image data 216, including one or more particles within the set of frames, captured by the particle analysis system 602. For example, for a stationary system configuration (e.g., as depicted in FIG. 3), the particle analysis system can generate a 3-frame synchronized frame set as the particle(s) freefall through an imaging plane 306.

Frames in the synchronized frames sets include one or more particles in each frame. For example, frames can include multiple particles. The particle analysis program identifies, in each frame of the set of frames, a set of particles in the frame 604. For example, the program can perform particle segmentation and overlap analysis to identify and distinguish the different particles appearing in each frame.

The program determines, for the identified particles in the set of frames, matching particles appearing in the set of frames 606. In other words, the program tags particles appearing in the set of frames and identifies appearances of a same particle in the frames. For example, a particle in freefall through imaging plane 306 appears in two or more frames of the set of frames captured by respective cameras and the program can identify and tag the particle in the two or more frames as a same particle.

Determining matching particles includes using transformation matrices for each of the cameras of the image capture sub-system to relate a position of a particle in a camera coordinate system to a world coordinate system. In other words, pixels of a frame are related to a world coordinate system by a transformation matrix. For example, a dot product translation between image coordinates and a transformation matrix can be used to reconstruct each pixel from image coordinates to world coordinates. Particles identified in the frame are tagged to the three-dimensional space of the world coordinates.

In some implementations, the program can assign a confidence score that an appearance of a particle in a frame is a same particle appearing in a different frame of the synchronized set. For example, using a silhouette of the particle in the frame, principal component analysis is applied to obtain a primary axis of the particle and then L2-norm (i.e., the Euclidean norm) for the particle is compared to an L2-norm of another potential appearance of the particle to generate the confidence score. In other words, a silhouette area of the particle can be used as a proxy for determining a confidence score that a same particle appears in two or more frames of the synchronized set.

The program applies a normalization to the particles appearing in the frames 608. For example, the program can modify a contrast between the particle pixels and the non-particle pixels to be at least a threshold high-contrast. In some implementations, the program uses thresholding to remove all the non-particle pixels in the frames. For example, a nearest neighbor algorithm can be used to identify a group of pixels representing a silhouette of a particle. In another example, erosion and dilation operations can be applied to frames to remove noisy pixels. In some implementations, for the identified particles in the frames of the set of frames, the program generates bounding boxes around each particle and thresholding to generate binary particle silhouette images.

For each matching set of particle views, the program generates a reconstruction of the particle 610. In some implementations, reconstruction of the particle can include using shape completion algorithms to reconstruct a 3D mesh representative of the particle. Use of shape completion algorithms can include assumptions of symmetries along each of the captured axes, e.g., along each of the three axes. For example, if three ellipses are captured along three axes, an assumption is made that the particle is an ellipsoid.

In some implementations, particle reconstruction includes using a Cauchy surface area formula, which states that the average area of a projection of a convex body is equal to its surface area up to a multiplicative constant in the dimension. For example, a multiplicative constant is four where a surface area can be approximately reconstructed by adding four (different) views of the surface of the particle. The program generates a particle surface area estimate by determining a surface area of each of the views of the particle in the captured frames and averaging the surface areas to generate a normalized surface area. For example, using three views of the particle, the program generates a normalized surface area from the three surface areas appearing in three views of the particle. The program calculates a particle volume, for example, using a different model or using the same model by adding an additional prediction head for particle volume. The normalized surface area is multiplied by a constant in the dimension, e.g., by 4 to generate the estimated particle surface area of the reconstructed particle.

In some implementations, particle reconstruction includes using a geometric coefficient C_{g} and calculating the specific surface area S of the particle by equation (1):

$$S = {C}_{g} X 〈 {P}_{s} 〉 = 1.35 x 〈 {P}_{s} 〉 \qquad ( 1 )$$

where S is the specific surface area of the particle, C_{g} is the geometric coefficient 1.35 and <Pₛ> is a normalized (e.g., averaged) specific perimeter of the particle (e.g., an average of perimeter/area of each view of the particle). For example, the program can calculate a normalized specific perimeter of three views of the particle by extracting, for each view of the particle, a perimeter and a surface area. The program can calculate, from the extracted perimeter and surface area, a specific perimeter for each view of the particle, and can generate a normalized specific perimeter by averaging the values of specific perimeter calculated for each view of the particle. The program calculates a specific surface area of the particle by multiplying the normalized specific perimeter by the geometric coefficient, e.g., 1.35.

In some implementations, the particle analysis program can implement a combination of Cauchy surface area formula and geometric coefficient-based methods described above. For example, the program can use an (e.g., weighted) average of the two values output by each method to produce a refined value of the specific surface area of the reconstructed particle.

The particle analysis program generates a characterization profile of the particle from the particle reconstruction 612. The particle analysis program can generate a characterization profile from the particle reconstruction based in part on, for example, a user input request. For example, a user can request a sieving distribution for the particles characterized by the particle analysis system. In another example, a user can request a specific area distribution curve for the particles characterized by the particle analysis system.

In some implementations, the program generates a characterization profile of the particles as particle size distribution (e.g., sieving distribution). For each reconstructed particle, the program designates a saving sieve (e.g., a sieve that will retain the particle) based on a surface area of the particle, e.g., extracted using the Cauchy surface area formula described above. The program can determine a retaining sieve based on a mesh size of the sieve and the surface area of the particle. The program can then sum respective volumes retained by each sieve by mesh size to obtain a weight percent retained per sieve.

In any case, the program can determine an updated composition of a mixture, e.g., an update to a batching recipe to obtain target material properties, in response to the characterization profile of the particles 614. As described above, the characterized sample can be a representative sample of dry ingredients of a material preparation system 100, where the characterization profile of the representative sample can be used to update the recipe to achieve a set of desired material properties, e.g., rheological properties.

In some implementations, particle analysis program utilizes a trained machine-learned model to predict, from a surface area of the particle, a retaining sieve mesh size for the particle. For example, the program can use a bounding box method, forming a bounding boxing around each view of the reconstructed particle and simulate (e.g., using a physics simulation engine) to see if the particle will pass through with a threshold amount of agitation.

In some implementations, the program can output, e.g., in a graphical user interface, a visual representation of the characterization profile, e.g., particle size distribution and/or sieving distribution. For example, the program can generate and present graphical representations of the particle size distribution and/or sieving distribution for a user on a user device.

In some implementations, the program generates a characterization profile of the particles as a specific surface area distribution curve. The program calculates a specific surface area of the characterized particles of the particle stream as:

$${\sum }_{i}^{N} \frac{{SA}_{i}}{{V}_{i}} = SSA ( N ) \qquad ( 2 )$$

Where SAᵢ is the specific surface area of a particle, Vᵢ, is the volume of the particle, and the specific surface area (SSA) all characterized particles Nis a summation thereof. The program can use a physics model to determine relationships between specific surface area distributions and other rheological properties. For example, the particle analysis program can determine that a first particle distribution having a 1 m²/m³ specific surface area has a first set of rheological characteristics and a second particle distribution has 1.1 m²/m³ has a second, different set of rheological characteristics. The characterization profile output from the particle analysis program, e.g., the specific surface area of the N particles, can be provided as input to the material preparation system 100. The material preparation system 100 can receive the specific surface area distribution and implement one or more adjustments in response. For example, batch recipes, batch ingredient ratios, preparation methods, or the like, can be adjusted in response to the characterization of the particles of the particle stream output by the particle analysis system 200.

In some implementations, the program can calculate a variance relative to the determined particle distribution where each particle size includes a confidence interval. For example, the program determines, for each particle a retaining mesh for the particle and an associated variance in the determination. Using the methods described above, the program can calculate a variance for an estimated particle size distribution with a variance of about ~0.1% or less, e.g., where a specific surface area for N particles is calculated as a sum of each specific surface area per particle. By determining the specific surface area of the collection of N particles using equation (2) rather than as a summation of surface area of all particles divided by a summation of all volumes of all particles, the program can calculate a specific surface area distribution of the collection of particles while significantly reducing a variance in the measurement, e.g., reduction in the error factor of the calculation.

In some implementations, the particle analyzing program can use data driven approaches to performed particle reconstruction and/or to generate characterizations of particles processed by the particle analyzing system. Generally, the program can use one or more neural networks to receive the multiple frames including a respective silhouette of a particle as input and generate predictions about the particle characteristics as output, e.g., to predict a specific surface area of the particle as output. In any case, the program can concatenate the frames, e.g., 3N frames, including the identified particles and provide the frames into the one or more neural networks. In one example, if N=1, a feed-forward neural network can be implemented, and if N>1, a convolutional neural network can be implemented.

In some implementations, one or more neural networks include a regression model trained on ground truth data. The ground truth data, e.g., training data 228, can be generated using “particles” of known characteristics. For example, dummy particles (e.g., ball bearings, 3D printed particles of known shape) can be used to generate ground truth data through a particle analyzing system for training the neural network. In some implementations, a synthetic particle generation engine can create training data, e.g., training data 228, including virtual particles having known characteristics (e.g., known specific surface areas) and apply ray tracing to create multiple orthogonal silhouettes. The synthetic data can be used to train the regression model to predict the specific surface area of a particle from the captured silhouettes of the particle.

In another example, the program can use a generative adversarial neural network (GANN). A generative model can depend in part on the types of objects that are being used, where the model is structured to be substantially general to cover a distribution of particles. The first neural network of the GANN can receive, as input, the multiple frames including a respective silhouette of a particle as input and generate a prediction of a particle reconstruction for the particle as an intermediary output, where a second neural network can determine, for the particle reconstruction prediction, whether the generated particle reconstruction of the particle is a natural or unnatural (e.g., realistic or unrealistic) particle. A realistic (natural) particle can be defined, for example, as falling within a threshold range of dimensions, ratio of dimensions (e.g., dimension along various axes), symmetries, convex/concave features, etc. The output of the second neural network (e.g., realistic vs unrealistic) can be used to refine the particle reconstruction of the particle from the input silhouettes by the first neural network.

### Calibration of the Particle Analysis System

In some implementations, a calibration process can be used for the particle analysis system such that the cameras of the system capture at least one particle in common in each synchronized image set. From the calibration process, camera coordinates for each camera of the image capture sub-system is mapped to a shared set of world coordinates such that locations of the cameras and light sources are known, and a set of transformation matrices are generated for the cameras to translate camera coordinates to world coordinates a of three-dimensional space of the particle analysis system.

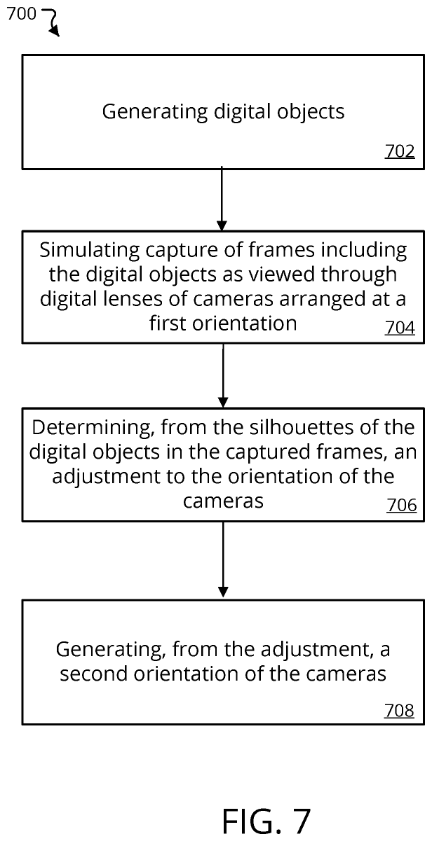

In some implementations, a calibration process is used to determine respective angles of orientation of the cameras with respect to each other and the particle stream. For example, orthogonal (90) angle orientations, angles of orientation between 70-90 degrees, angles of orientation at less than 70 degrees, etc. Generally, the program generates a simulated model of the particle analyzing system to determine (e.g., optimize) how to orient the cameras of a particle analyzing system. For example, as particles become increasingly less regular (e.g., less spherical or non-regular), the closer to 90-degree angles of camera orientation may be desirable. FIG. 7 is a flow diagram of an example process 700 of the particle analyzing system. The program generates digital object(s) 702 and simulates capturing frames including the digital object(s) as viewed through digital lenses of cameras arranged at a first orientation 704. The program determines, from the silhouettes of the digital object(s) in the captured frames, an adjustment to the orientation of the cameras 706 and generates a second orientation of the cameras 708. For example, the angles of orientation between the cameras of the image capture sub-system can be 90 degrees or less. In some instances, e.g., when using the system to characterize non-regular objects of interest, an angle of orientation between the cameras can be selected to be closer to or equal to 90 degrees. In some instances, e.g., when using the system to characterize regular objects of interest (e.g., spherical), an angle of orientation between the cameras of the image capture sub-system can be selected to be less than 90 degrees. In other words, as the objects of interest become increasingly irregular in shape, an angle of orientation between the cameras can be selected to be closer to or equal to 90 degrees.

In some implementations, a calibration of the orientation of the cameras of the particle analyzing system includes arranging the cameras in a first, initial orientation with respect to the imaging region and dropping test objects of known dimensions in freefall through the imaging region. The program can analyze the captured frames by the cameras in the first orientation and generate a calibration adjustment to one or more orientations of the cameras in response.

In some implementations, a calibration of the cameras of the particle analyzing system includes synchronizing the cameras of the image capture sub-system to reduce (e.g., prevent) capturing a rotation of the particle in the captured frames by two or more cameras at an imaging plane. For example, the IEEE 1588-2008 standard for a precision clock synchronization protocol for networked measurement and control systems can be applied.

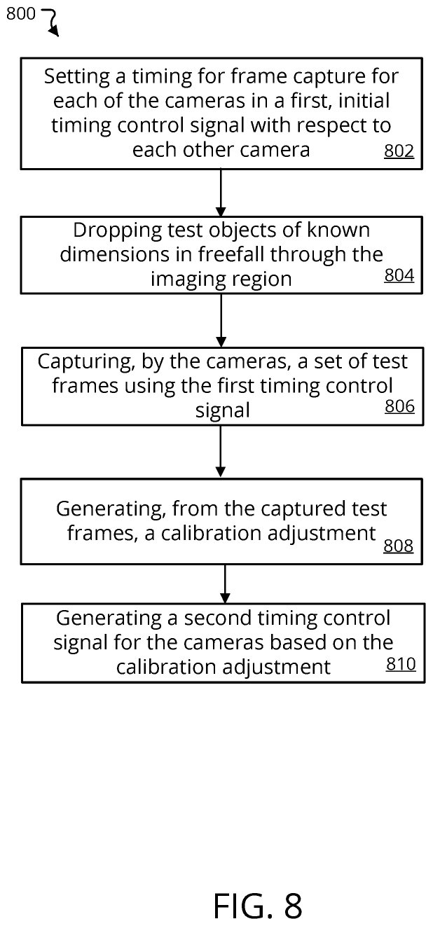

FIG. 8 is a flow diagram of an example process 800 of the particle analyzing system. Calibration of the synchronization of the cameras of the particle analyzing system includes setting a timing for frame capture for each of cameras in a first, initial timing control signal (e.g., by control system 102) with respect to each other camera 802, dropping test objects of known dimensions in freefall through the imaging region 804, and capturing by the cameras a set of test frames 806. The program can generate a calibration adjustment from the captured test frames by the cameras using the first timing control signal 808 and generate a second timing control signal for the cameras based on the calibration adjustment 810.

### Additional Embodiments

Although described in this specification as systems and methods for characterizing particles, e.g., sand or other ingredients of concrete, the methods and systems described herein can be applied generally to characterize other roughly spherical or particulate matter. For example, other industries for which the systems and methods can be applied include food processing, pharmaceuticals, concrete, or other industries involving particulate matter suspended in solution.

In one embodiment, the methods and system can be applied to characterize droplets of paint, e.g., during a batching process. For example, a droplet of paint can be isolated and dried, where the particles/powder of the dried droplet composition can be characterized using the systems and methods described above to better understand the properties of the dry ingredients. In some implementations, a pre-processing step can be used to prepare the paint by spin coating and then evaporating a solvent of the paint composition. Image data can be collected (e.g., periodically) during the drying process to generate an understanding of how the paint dries.

In another embodiment, the methods and system can be applied to characterize shape and specific area of drug particles in pharmaceutical research and development and/or manufacturing, where characteristics of the drug particles may impact delivery and/or impact of the drug.

In another embodiments, the methods and system can be applied to characterize shape and specific surface area to better understand and model glassy and viscous mechanics of asphalt composites.

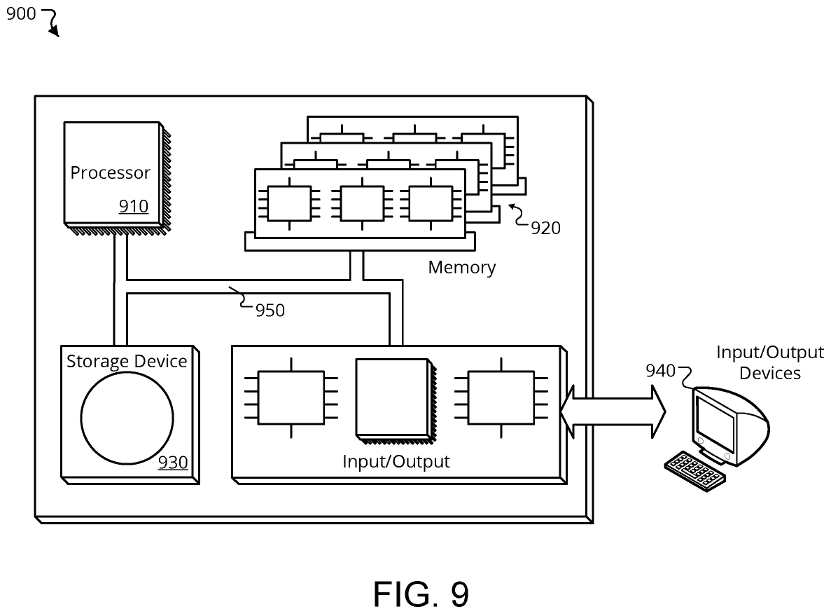

FIG. 9 is a schematic diagram of a computer system 900. The system 900 can be used to carry out the operations described in association with any of the computer-implemented methods described previously, according to some implementations, for example, the operations described with respect to particle analysis program 224. In some implementations, computing systems and devices and the functional operations described in this specification can be implemented in digital electronic circuitry, in tangibly-embodied computer software or firmware, in computer hardware, including the structures disclosed in this specification (e.g., system 900) and their structural equivalents, or in combinations of one or more of them. The system 900 is intended to include various forms of digital computers, such as laptops, desktops, workstations, personal digital assistants, servers, blade servers, mainframes, and other appropriate computers, including vehicles installed on base units or pod units of modular vehicles. The system 900 can also include mobile devices, such as personal digital assistants, cellular telephones, smartphones, and other similar computing devices. Additionally, the system can include portable storage media, such as, Universal Serial Bus (USB) flash drives. For example, the USB flash drives may store operating systems and other applications. The USB flash drives can include input/output components, such as a wireless transducer or USB connector that may be inserted into a USB port of another computing device.

The system 900 includes a processor 910, a memory 920, a storage device 930, and an input/output device 940. Each of the components 910, 920, 930, and 940 are interconnected using a system bus 950. The processor 910 is capable of processing instructions for execution within the system 900. The processor may be designed using any of a number of architectures. For example, the processor 910 may be a CISC (Complex Instruction Set Computers) processor, a RISC (Reduced Instruction Set Computer) processor, or a MISC (Minimal Instruction Set Computer) processor.

In one implementation, the processor 910 is a single-threaded processor. In another implementation, the processor 910 is a multi-threaded processor. The processor 910 is capable of processing instructions stored in the memory 920 or on the storage device 930 to display graphical information for a user interface on the input/output device 940.

The memory 920 stores information within the system 900. In one implementation, the memory 920 is a computer-readable medium. In one implementation, the memory 920 is a volatile memory unit. In another implementation, the memory 920 is a non-volatile memory unit.

The storage device 930 is capable of providing mass storage for the system 900. In one implementation, the storage device 930 is a computer-readable medium. In various different implementations, the storage device 930 may be a floppy disk device, a hard disk device, an optical disk device, or a tape device.

The input/output device 940 provides input/output operations for the system 900. In one implementation, the input/output device 940 includes a keyboard and/or pointing device. In another implementation, the input/output device 940 includes a display unit for displaying graphical user interfaces.

The features described can be implemented in digital electronic circuitry, or in computer hardware, firmware, software, or in combinations of them. The apparatus can be implemented in a computer program product tangibly embodied in an information carrier, e.g., in a machine-readable storage device for execution by a programmable processor; and method steps can be performed by a programmable processor executing a program of instructions to perform functions of the described implementations by operating on input data and generating output. The described features can be implemented advantageously in one or more computer programs that are executable on a programmable system including at least one programmable processor coupled to receive data and instructions from, and to transmit data and instructions to, a data storage system, at least one input device, and at least one output device. A computer program is a set of instructions that can be used, directly or indirectly, in a computer to perform a certain activity or bring about a certain result. A computer program can be written in any form of programming language, including compiled or interpreted languages, and it can be deployed in any form, including as a stand-alone program or as a sub-system, component, subroutine, or other unit suitable for use in a computing environment.

Suitable processors for the execution of a program of instructions include, by way of example, both general and special purpose microprocessors, and the sole processor or one of multiple processors of any kind of computer. Generally, a processor will receive instructions and data from a read-only memory or a random access memory or both. The essential elements of a computer are a processor for executing instructions and one or more memories for storing instructions and data. Generally, a computer will also include, or be operatively coupled to communicate with, one or more mass storage devices for storing data files; such devices include magnetic disks, such as internal hard disks and removable disks; magneto-optical disks; and optical disks. Storage devices suitable for tangibly embodying computer program instructions and data include all forms of non-volatile memory, including by way of example semiconductor memory devices, such as EPROM, EEPROM, and flash memory devices; magnetic disks such as internal hard disks and removable disks; magneto-optical disks; and CD-ROM and DVD-ROM disks. The processor and the memory can be supplemented by, or incorporated in, ASICs (application-specific integrated circuits).

To provide for interaction with a user, the features can be implemented on a computer having a display device such as a CRT (cathode ray tube) or LCD (liquid crystal display) monitor for displaying information to the user and a keyboard and a pointing device such as a mouse or a trackball by which the user can provide input to the computer. Additionally, such activities can be implemented via touchscreen flat-panel displays and other appropriate mechanisms.

The features can be implemented in a computer system that includes a back-end component, such as a data server, or that includes a middleware component, such as an application server or an Internet server, or that includes a front-end component, such as a client computer having a graphical user interface or an Internet browser, or any combination of them. The components of the system can be connected by any form or medium of digital data communication such as a communication network. Examples of communication networks include a local area network (“LAN”), a wide area network (“WAN”), peer-to-peer networks (having ad-hoc or static members), grid computing infrastructures, and the Internet.

The computer system can include clients and servers. A client and server are generally remote from each other and typically interact through a network, such as the described one. The relationship of client and server arises by virtue of computer programs running on the respective computers and having a client-server relationship to each other.

### Examples

Although the disclosed inventive concepts include those defined in the attached claims, it should be understood that the inventive concepts can also be defined in accordance with the following embodiments.

In addition to the embodiments of the attached claims and the embodiments described above, the following numbered embodiments are also innovative.

### a System for Particle Analysis

Embodiment 1 is a system for particle analysis comprising:
   a hopper configured to dispense particles along an axis, wherein the particles dispensed by the hopper freefall along the axis through an imaging region;
   an illumination sub-system comprising at least one light source arranged at a first location with respect to the axis and configured to illuminate the imaging region;
   an image capture sub-system comprising at least one image capture device including a telecentric lens and arranged at a second location with respect to the axis and opposite from the illumination sub-system, the image capture sub-system configured to align a focal plane of the at least one image capture device with the axis within the imaging region,
   wherein, when a particle of the particles freefalls along the axis through the imaging region, the particle is illuminated by the illumination sub-system and wherein the image capture sub-system is configured to capture images of at least three silhouettes of the particle within the imaging region.
Embodiment 2 is the system of Embodiment 1, wherein:
the image capture sub-system comprises three image capture devices, the image capture devices arranged at respective angles with respect to each other, and wherein each image capture device is arranged opposite the illumination sub-system with respect to the axis.
Embodiment 3 is the system of Embodiment 2, wherein:
the three image capture devices are arranged orthogonally with respect to each other, and wherein each image capture device is arranged at a 45-degree angle with respect to the axis.
Embodiment 4 is the system of Embodiment 2, wherein:
the three image capture devices are arranged at an angle less than 90 degrees with respect to each other.
Embodiment 5 is the system of any previous Embodiment, further comprising:
for each image capture device of the three image capture devices, a respective filter,
wherein each filter is selective of at least one of (A) a different wavelength emission spectrum and (B) a different polarization angle of light emitted from the at least one light source.
Embodiment 6 is the system of Embodiment 5, wherein:
each image capture sub-system captures the silhouettes from different perspectives of the particle.
Embodiment 7 is the system of Embodiment 5, wherein:
the illumination sub-system comprises three light sources, each light source configured to emit light of at least one of (A) a different wavelength spectrum and (B) a different polarization angle.
Embodiment 8 is the system of Embodiment 7, wherein:
each light source of the three light sources is paired with a corresponding image capture device of the three image capture devices and opposite the axis from the paired image capture device, and
wherein the filter of the corresponding image capture device is selected to filter a threshold emission from each other light source that is not paired with the image capture device.
Embodiment 9 is the system of Embodiment 8, wherein:
the imaging region is an imaging plane perpendicular to the axis, and wherein focal planes of three image capture devices are aligned to an intersection of the axis with the imaging plane.
Embodiment 10 is the system of any previous Embodiment, wherein:
the image capture sub-system comprises four image capture devices, the image capture devices arranged at respective angles with respect to each other, and wherein each image capture device is arranged opposite the illumination sub-system with respect to the axis.
Embodiment 11 is the system of any previous Embodiment, wherein:
the image capture sub-system further comprises a scanner arranged with respect to the at least one image capture device to align a focal plane of the at least one image capture device along the axis with the imaging region.
Embodiment 12 is the system of Embodiment 11, wherein:
the hopper is configured to induce a tumbling movement in the particle as the particle freefalls along the axis through the imaging region.
Embodiment 13 is the system of Embodiment 12, wherein:
the scanner is configured to, when the particle of the particles freefalls along the axis through the imaging region, adjust an alignment of the focal plane of the image capture device with a current location of the particle along the axis to capture the at least three silhouettes of the particle, and
wherein each silhouette is captured at a different location of the particle along the axis.
Embodiment 14 is the system of Embodiment 13, wherein:
the illumination sub-system further comprises a line scanner and a beam splitter, wherein the line scanner is arranged respect to the axis and configured to capture high speed measurements of the current location of the particle as the particle freefalls through the imaging region.
Embodiment 15 is the system of Embodiment 13, wherein:
the at least one light source of the illumination sub-system is a diffuse light source.
Embodiment 16 is the system of any previous Embodiment, further comprising:
two or more image capture sub-systems arranged at respective angles with respect to each other, and wherein each image capture sub-system is arranged opposite the illumination sub-system with respect to the axis.
Embodiment 17 is the system of Embodiment 16, wherein:
the two or more image capture sub-systems are configured to capture, at three or more locations along the axis within the imaging region, a set of synchronized frames including the silhouette of the particle as the particle freefalls through the imaging region.
Embodiment 18 is the system of any Embodiment 15 to 17, wherein:
the diffuse light source comprises a plurality of light emitting diodes, and wherein the plurality of light sources are configured to emit a same number of (A) different wavelength spectra or (B) different polarization angles as the two or more image capture sub-systems of the system.
Embodiment 19 is the system of any Embodiment 15 to 18, wherein:
each of the two or more image capture sub-systems of the system further comprises a filter corresponding to at least one of (A) the different wavelength spectra and (B) the different polarization angles of the diffuse light source.
Embodiment 20 is the system of any previous Embodiment, further comprising:
a controller in data communication with the illumination sub-system, image capture sub-system, and the hopper and configured to perform operations comprising:
receiving image data of a plurality of particles representative of a particulate ingredient of a mixture, wherein the image data comprises sets of frames, and wherein each set of frames includes silhouettes of one or more particles captured within the set of frames;
for each set of frames
   identifying, in each frame of the set of frames, a silhouette of at least one particle in the frame;
   matching respective silhouettes from each frame of the set of frames that represent a same particle appearing in each frame from different perspectives; and
   for the respective silhouettes corresponding to same particle
      generating a reconstruction of the particle from the respective silhouettes to obtain a reconstructed particle; and
      extracting from the reconstruction of the particle, one or more characteristics of the particle;
generating, from the reconstructed particles of the plurality of particles, a characteristic profile of the plurality of particles; and
determining, in response to the characteristic profile, an update to a composition of the mixture.

### Method for Characterizing a Particulate Ingredient of a Mixture

Embodiment 21 is a method for characterizing a particulate ingredient of a mixture, the method comprising:
receiving image data of a plurality of particles representative of the particulate ingredient of the mixture, wherein the image data comprises sets of frames, and wherein each set of frames includes silhouettes of one or more particles captured within the set of frames;
for each set of frames
   identifying, in each frame of the set of frames, a silhouette of at least one particle in the frame;
   matching respective silhouettes from each frame of the set of frames that represent a same particle appearing in each frame from different perspectives; and
   for the respective silhouettes corresponding to same particle
      generating a reconstruction of the particle from the respective silhouettes to obtain a reconstructed particle; and
      extracting from the reconstruction of the particle, one or more characteristics of the particle;
generating, from the reconstructed particles of the plurality of particles, a characteristic profile of the plurality of particles; and
determining, in response to the characteristic profile, an update to a composition of the mixture.
Embodiment 22 is the method of Embodiment 21, wherein:
when frames in a set of frames include images of multiple silhouettes, identifying, in each from of the set of frames, comprises identifying particular silhouettes, each corresponding to each representing a different particle, and
wherein matching the respective silhouettes comprises matching each particular silhouette from each frame with a corresponding particular silhouette from the other frames that represents the same particle from a different perspective.
Embodiment 23 is the method of Embodiments 21 or 22, further comprising: configuring a particle analysis system, the configuring comprising:
arranging, at a first location with respect to an axis, an illumination sub-system comprising at least one light source configured to illuminate an imaging region; and
arranging, at a second location with respect to the axis and opposite from the illumination sub-system, an image capture sub-system comprising at least one image capture device, the image capture sub-system configured to align a focal plane of the at least one image capture device with the axis within the imaging region.
Embodiment 24 is the method of any previous Embodiment 21 to 23, further comprising:
generating, by a hopper, a particle stream comprising the plurality of particles, wherein particles of the particle stream freefall along the axis and through the imaging region of the particle analysis system;
illuminating, by the illumination sub-system, the imaging region; and
capturing the image data, when a particle of the plurality of particles freefalls along the axis through the imaging region, the image data comprising at least three silhouettes of the particle by the image capture sub-system.
Embodiment 25 is the method of any previous Embodiment 21 to 24, wherein:
arranging the at least one image capture device with respect to the axis comprises applying a virtual calibration process, the virtual calibration process comprising:
generating, by a computer program, digital objects;
simulating capture of frames including silhouettes of the digital objects as viewed through digital lenses of image capture devices arranged at a first orientation;
determining, from the silhouettes of the digital objects in the captured frames, an adjustment to the orientation of the image capture devices; and
generating, from the adjustment, a second orientation of the image capture devices.
Embodiment 26 is the method of any previous Embodiment 21 to 25, wherein:
the image capture sub-system comprises two or more image capture devices, and
wherein capturing the image data comprising the at least three silhouettes of the particle by the image capture sub-system further comprises application a calibration process, the calibration process comprising:
setting an initial timing control signal for frame capture for each image capture device of the two or more image capture devices, the initial timing control signal with respect to each other image capture device;
introducing test objects of known dimension in freefall along the axis and through the imaging region;
capturing, by the two or more image capture devices, a set of test frames using the initial timing control signal;
generating, from the captured test frames, a calibration adjustment; and
generating a second timing control signal for the image capture devices based on the calibration adjustment.
Embodiment 27 is the method of any previous Embodiment 21 to 26, wherein:
generating the reconstruction of the particle from the set of silhouettes comprises using (A) a Cauchy surface area formulation, (B) a geometric coefficient formulation, or (C) a combination thereof.
Embodiment 28 is the method of any previous Embodiment 21 to 27, wherein:
extracting from the reconstruction of the particle the characteristic of the particle comprises extracting a specific surface area of the particle.
Embodiment 29 is the method of Embodiment 28, wherein:
generating, from the reconstructed particles of the plurality of particles, the characteristic profile of the plurality of particles comprises:
generating a specific surface area distribution for the plurality of particles.
Embodiment 30 is the method of Embodiment 29, wherein:
generating, from the reconstructed particles of the plurality of particles, the characteristic profile of the plurality of particles comprises:
generating, from the specific surface areas of the plurality of particles, a sieving distribution profile for the plurality of particles.
Embodiment 31 is the method of Embodiment 30, wherein:
generating the sieving distribution profile comprises applying a trained machine-learned model to predict, from the specific surface areas of each of the plurality of particles, a retaining sieve mesh size for the particle.
Embodiment 32 is the method of any Embodiments 30 and 31, wherein generating the sieving distribution profile comprises:
forming, for each particle, a bounding boxing around each silhouette of the reconstructed particle; and
iteratively simulating, by a physics simulation engine and for increasing mesh sizes, whether the bounding box of particle will pass through the mesh size with a threshold amount of agitation.
Embodiment 33 is the method of any previous Embodiment 21 to 32, wherein:
matching, for the identified silhouettes of the particle in the set of frames, silhouettes corresponding to same particles appearing in the set of frames comprises:
applying a transformation matrix for each image capture device of the image capture sub-system to map a position of the silhouettes of the particle in the set of frames to a world coordinate system.
Embodiment 34 is the method of any previous Embodiment 21 to 33, further comprising:
assigning to each silhouette of the particle in the set of frames a confidence score that an appearance of the particle in a frame of the set of frames is a same particle appearing in a different frame of the frames.
Embodiment 35 is the method of any previous Embodiment 21 to 34, further comprising:
applying a normalization to the frames, wherein the normalization comprises modifying a contrast between first pixels corresponding to particles and second pixels corresponding to non-particles such that the contrast is at least a threshold high-contrast.
Embodiment 36 is the method of Embodiment 35, further comprising:
generating a bounding box around each particle and applying a thresholding to generate a binary particle silhouette image.
Embodiment 37 is the method of any previous Embodiment 21 to 36, wherein:
generating the reconstruction of the particle from the set of silhouettes and extracting from the reconstruction of the particle the characteristic of the particle comprises:
providing, to a machine-learned model, the set of silhouettes; and
receiving, from the machine-learned model, the characteristic of the particle.
Embodiment 38 is the method of Embodiment 37, wherein:
the machine-learned model comprises a trained regression model.
Embodiment 39 is the method of Embodiment 38, wherein:
training the trained regression model comprises:
generating synthetic data comprising virtual particles having known specific surface areas;
applying ray tracing to generate a plurality of orthogonal silhouettes of the virtual particles; and
training the regression model to predict a specific surface area of the virtual particles from captured silhouettes of the virtual particles.
Embodiment 40 is the method of Embodiment 37, wherein:
the machine-learned model comprises a generative adversarial neural network.
Embodiment 41 is a system comprising: one or more computers and one or more storage devices storing instructions that are operable, when executed by the one or more computers, to cause the one or more computers to perform the method of any one of claims 21 to 40.
Embodiment 42 is a computer storage medium encoded with a computer program, the program comprising instructions that are operable, when executed by data processing apparatus, to cause the data processing apparatus to perform the method of any one of claims 21 to 40.

While this specification contains many specific implementation details, these should not be construed as limitations on the scope of any inventions or of what may be claimed, but rather as descriptions of features specific to particular implementations of particular inventions. Certain features that are described in this specification in the context of separate implementations can also be implemented in combination in a single implementation. Conversely, various features that are described in the context of a single implementation can also be implemented in multiple implementations separately or in any suitable subcombination. Moreover, although features may be described above as acting in certain combinations and even initially claimed as such, one or more features from a claimed combination can in some cases be excised from the combination, and the claimed combination may be directed to a subcombination or variation of a subcombination.

Similarly, while operations are depicted in the drawings in a particular order, this should not be understood as requiring that such operations be performed in the particular order shown or in sequential order, or that all illustrated operations be performed, to achieve desirable results. In certain circumstances, multitasking and parallel processing may be advantageous. Moreover, the separation of various system components in the implementations described above should not be understood as requiring such separation in all implementations, and it should be understood that the described program components and systems can generally be integrated together in a single software product or packaged into multiple software products.

Thus, particular implementations of the subject matter have been described. Other implementations are within the scope of the following claims. In some cases, the actions recited in the claims can be performed in a different order and still achieve desirable results. In addition, the processes depicted in the accompanying figures do not necessarily require the particular order shown, or sequential order, to achieve desirable results. In certain implementations, multitasking and parallel processing may be advantageous.

As used herein, the term “ready mix” refers to concrete that is batched for delivery from a central plant instead of being mixed on a job site. Typically, a batch of ready mix is tailor-made according to the specifics of a particular construction project and delivered in a plastic condition, usually in cylindrical trucks often referred to as “concrete mixers”.

As used herein, the term “real-time” refers to transmitting or processing data without intentional delay given the processing limitations of a system, the time required to accurately obtain data, and the rate of change of the data. Although there may be some actual delays, the delays are generally imperceptible to a user.

What is claimed is:

1. A method for characterizing a particulate ingredient of a mixture, the method comprising:
receiving image data of a plurality of particles representative of the particulate ingredient of the mixture, wherein the image data comprises sets of frames, and wherein each set of frames includes silhouettes of one or more particles captured within the set of frames;
for each set of frames
identifying, in each frame of the set of frames, a silhouette of at least one particle in the frame;
matching respective silhouettes from each frame of the set of frames that represent a same particle appearing in each frame from different perspectives; and
for the respective silhouettes corresponding to same particle;
generating a reconstruction of the particle from the respective silhouettes to obtain a reconstructed particle; and
extracting from the reconstruction of the particle, one or more characteristics of the particle;
generating, from the reconstructed particles of the plurality of particles, a characteristic profile of the plurality of particles; and
determining, in response to the characteristic profile, an update to a composition of the mixture.

2. The method of claim 1, wherein, when frames in a set of frames include images of multiple silhouettes, identifying, in each from of the set of frames, comprises
identifying particular silhouettes, each corresponding to each representing a different particle, and
wherein matching the respective silhouettes comprises matching each particular silhouette from each frame with a corresponding particular silhouette from the other frames that represents the same particle from a different perspective.

3. The method of claim 1, further comprising configuring a particle analysis system, the configuring comprising:
arranging, at a first location with respect to an axis, an illumination sub-system comprising at least one light source configured to illuminate an imaging region; and
arranging, at a second location with respect to the axis and opposite from the illumination sub-system, an image capture sub-system comprising at least one image capture device, the image capture sub-system configured to align a focal plane of the at least one image capture device with the axis within the imaging region.

4. The method of claim 3, further comprising:
generating, by a hopper, a particle stream comprising the plurality of particles, wherein particles of the particle stream freefall along the axis and through the imaging region of the particle analysis system;
illuminating, by the illumination sub-system, the imaging region; and
capturing the image data, when a particle of the plurality of particles freefalls along the axis through the imaging region, the image data comprising at least three silhouettes of the particle by the image capture sub-system.

5. The method of claim 3, wherein arranging the at least one image capture device with respect to the axis comprises applying a virtual calibration process, the virtual calibration process comprising:
generating, by a computer program, digital objects;
simulating capture of frames including silhouettes of the digital objects as viewed through digital lenses of image capture devices arranged at a first orientation;
determining, from the silhouettes of the digital objects in the captured frames, an adjustment to the orientation of the image capture devices; and
generating, from the adjustment, a second orientation of the image capture devices.

6. The method of claim 4, wherein the image capture sub-system comprises two or more image capture devices, and
wherein capturing the image data comprising the at least three silhouettes of the particle by the image capture sub-system further comprises application a calibration process, the calibration process comprising:
setting an initial timing control signal for frame capture for each image capture device of the two or more image capture devices, the initial timing control signal with respect to each other image capture device;
introducing test objects of known dimension in freefall along the axis and through the imaging region;
capturing, by the two or more image capture devices, a set of test frames using the initial timing control signal;
generating, from the captured test frames, a calibration adjustment; and
generating a second timing control signal for the image capture devices based on the calibration adjustment.

7. The method of claim 1, wherein generating the reconstruction of the particle from the set of silhouettes comprises using (A) a Cauchy surface area formulation, (B) a geometric coefficient formulation, or (C) a combination thereof.

8. The method of claim 1, wherein extracting from the reconstruction of the particle the characteristic of the particle comprises extracting a specific surface area of the particle.

9. The method of claim 8, wherein generating, from the reconstructed particles of the plurality of particles, the characteristic profile of the plurality of particles comprises:
generating a specific surface area distribution for the plurality of particles.

10. The method of claim 9, wherein generating, from the reconstructed particles of the plurality of particles, the characteristic profile of the plurality of particles comprises:
generating, from the specific surface areas of the plurality of particles, a sieving distribution profile for the plurality of particles.

11. The method of claim 10, wherein generating the sieving distribution profile comprises applying a trained machine-learned model to predict, from the specific surface areas of each of the plurality of particles, a retaining sieve mesh size for the particle.

12. The method of claim 10, wherein generating the sieving distribution profile comprises:
forming, for each particle, a bounding boxing around each silhouette of the reconstructed particle; and
iteratively simulating, by a physics simulation engine and for increasing mesh sizes, whether the bounding box of particle will pass through the mesh size with a threshold amount of agitation.

13. The method of claim 3, wherein matching, for the identified silhouettes of the particle in the set of frames, silhouettes corresponding to same particles appearing in the set of frames comprises:
applying a transformation matrix for each image capture device of the image capture sub-system to map a position of the silhouettes of the particle in the set of frames to a world coordinate system.

14. The method of claim 13, further comprising: assigning to each silhouette of the particle in the set of frames a confidence score that an appearance of the particle in a frame of the set of frames is a same particle appearing in a different frame of the frames.

15. The method of claim 1, further comprising applying a normalization to the frames, wherein the normalization comprises modifying a contrast between first pixels corresponding to particles and second pixels corresponding to non-particles such that the contrast is at least a threshold high-contrast.

16. The method of claim 15, further comprising generating a bounding box around each particle and applying a thresholding to generate a binary particle silhouette image.

17. The method of claim 1, wherein generating the reconstruction of the particle from the set of silhouettes and extracting from the reconstruction of the particle the characteristic of the particle comprises:
providing, to a machine-learned model, the set of silhouettes; and
receiving, from the machine-learned model, the characteristic of the particle.

18. The method of claim 17, wherein the machine-learned model comprises a trained regression model.

19. The method of claim 18, wherein training the trained regression model comprises:
generating synthetic data comprising virtual particles having known specific surface areas;
applying ray tracing to generate a plurality of orthogonal silhouettes of the virtual particles; and
training the regression model to predict a specific surface area of the virtual particles from captured silhouettes of the virtual particles.

20. The method of claim 17, wherein the machine-learned model comprises a generative adversarial neural network.
